(19) United States
(12) Patent Application Publication
(10) Pub. No.: US 2023/0364152 A1
(43) Pub. Date: Nov. 16, 2023

(54) COMPOSITIONS AND METHODS OF USE FOR INFUSIBLE EXTRACELLULAR MATRIX

(71) Applicant: The Regents of the University of California, Oakland, CA (US)

(72) Inventors: Karen L. Christman, San Diego, CA (US); Ryan Middleton, San Diego, CA (US); Raymond Wang, La Jolla, CA (US); Mark Hepokoski, Capistrano Beach, CA (US); Martin Spang, San Diego, CA (US)

(21) Appl. No.: 18/245,128

(22) PCT Filed: Sep. 14, 2021
(86) PCT No.: PCT/US2021/050181

§ 371 Date: Mar. 13, 2023

Related U.S. Application Data
(60) Provisional application No. 63/077,803, filed on Sep. 14, 2020.

(51) Int. Cl. A61K 35/34; A61P 9/10

(52) U.S. Cl. CPC A61K 35/34; A61P 9/10

(57) ABSTRACT

Compositions and methods of using infusible extracellular matrix (iECM) as a treatment for vascular injury or tissue injury.

### CROSS REFERENCE TO RELATED APPLICATIONS

This application claims the priority benefit of U.S. Provisional Application No. 63/077,803 filed Sep. 14, 2020, which application is incorporated herein by reference.

### GOVERNMENT SPONSORSHIP

This invention was made with government support under grant no. HL113468 awarded by the National Institutes of Health (NIH). The government has certain to rights in the invention.

### TECHNICAL FIELD

The present invention relates to compositions and methods of use for infusible extracellular matrix.

### BACKGROUND

Severe systemic inflammatory conditions, such as in sepsis, Acute Respiratory Distress Syndrome (ARDS), and COVID-19 infection, can lead to multi-organ damage. Reduced endothelial barrier function facilitates systemic spread of pathogens, and increases immune activation and inflammation in organs outside of the primary infection site. Current standards of care for systemic inflammation management, including fluid resuscitation and antibiotic administration, do not directly address or attempt to prevent multi-organ inflammation.

Disease conditions eliciting dysregulated systemic inflammation such as sepsis, acute respiratory distress syndrome (ARDS), and the recent SARS-CoV-2 virus, have led to millions of deaths and patients with lingering tissue damage post-acute symptom recovery worldwide. Similar pathophysiology of hyperinflammation and increased vascular permeability across multiple organs, as a result of high pathogen burden or an inability to control infection, risks potential development of severe downstream comorbidities and multi-organ failure. Therapeutic standards for early systemic inflammation typically involve fluid resuscitation in parallel with antibiotics or antivirals. Monoclonal antibodies, like tocilizumab or bamlanivimab, are becoming a notable area of development to target specific mediators of systemic inflammation, whether it is inflammatory receptors and cytokines or the infectious substances themselves.

A need remains for improved compositions and methods for treating multi-organ damage, vascular injury, leaky vasculature, increased vascular permeability, pulmonary arterial hypertension, lung injury, cardiac injury, or other tissue damage.

### SUMMARY OF THE INVENTION

In embodiments, the present disclosure provides compositions and methods for treatment of vascular injury, leaky vasculature, including but not limited to reducing or blocking the leaky vasculature permeability, reducing the infiltration immune cells, exudate, reactive oxygen species, inflammatory cytokines, growth factors, exosomes, or any proteins, particles, or molecules that may be deleterious to tissue function, result in negative tissue remodeling, or disease progression. Infusible extracellular matrix (ECM) administration, such as by infusion, can be used as treatments for injuries involving leaky vasculature, including tumors, myocardial infarction, stroke, and other ischemic conditions.

In embodiments, the present disclosure provides that the infusible ECM is combined with cells, peptides, proteins, DNA, drugs, nanoparticles, antibiotics, growth factors, nutrients, exosomes and extracellular vesicles, survival promoting additives, proteoglycans, and/or glycosaminoglycans.

In embodiments, the present disclosure provides that the composition of infusible extracellular matrix is derived from human, animal, embryonic, and/or fetal tissue sources. In embodiments, the present disclosure provides that the composition of infusible extracellular matrix is derived from heart, brain, bladder, small intestine, skeletal muscle, kidney, liver, lung, bronchioles, blood vessels, and other tissues/organs tissue sources.

In embodiments, the present disclosure provides a method for treating pulmonary arterial hypertension (PAH) comprising infusing in a subject in need with PAH an effective amount of a composition comprising infusible decellularized extracellular matrix derived from muscle, lung tissue or other tissues.

In embodiments, the present disclosure provides that the infusible ECM composition does not gel in vitro or inside the blood in vivo, but does gel when administered directly into tissue in vivo.

In embodiments, the present disclosure provides that said composition is delivered intravenously by infusion. In embodiments, the present disclosure provides that said composition is delivered by intracoronary infusion with a balloon infusion catheter. In embodiments, the present disclosure provides that said composition transitions to a gel form in tissue after delivery. In embodiments, the present disclosure provides that said composition transitions to form a coating on the endothelium of injured blood vessels after delivery. In embodiments, the present disclosure provides that said composition degrades within one to 14 days following injection or infusion.

In embodiments, the present disclosure provides that the injection or infusion of said composition repairs damage to cardiac muscle sustained by said subject, such as a right ventricle heart failure, associated with PAH. In embodiments, the present disclosure provides that said effective amount is an amount that increases blood flow, increases viable tissue mass, or induces new vascular formation in the area of the injection or infusion of the subject. In embodiments, the present disclosure provides that said effective amount is an amount that promotes cell survival, reduces inflammation, and repairs damaged vasculature in the area of the injection or infusion of the subject.

In embodiments, the present disclosure provides prophylactic protection against acute respiratory distress syndrome (ARDS) with or without ventilator induced lung injury (VILI). In embodiments, the present disclosure provides delivery of infusible ECM via intravenous infusion, lung lavage or aspiration by nebulization for the protection of alveolar epithelium as well as endothelial cells and other microvascular and vascular components within the lung, heart and peripheral organs and tissues. In embodiments, the present disclosure provides delivery of infusible material before, concurrently, or after tissue damage associated with ventilator injury, stomach content aspiration, or accidental or deliberate inspiration of environmental toxins.

In embodiments, the present disclosure provides methods for treatment of injury associated with lung damage, cardiac damage or multi-organ tissue damage occurring from systemic inflammation/cytokine storm or occurring from post-infection complications from a bacterial or viral source such as COVID-19, or an associated tissue disease comorbidity. In embodiments, the present disclosure provides methods for treatment of injury associated with COVID-19 within injured lungs when delivered intravenously or via intratracheal instillation.

### BRIEF DESCRIPTION OF THE DRAWINGS

FIGS. 1A to 1I are images showing gelation test of myocardial matrix (MM) and infusible extracellular matrix (iECM). Both solutions were imaged before incubation (together in FIGS. 1A, and separately in FIGS. 1B and 1C), 1 hour following to incubation at 37 degrees Celsius (together in FIG. 1D, and separately in FIGS. 1E and 1F), and 24 hours following incubation (together in FIG. 1G, and separately in FIGS. 1H and 1I). FIGS. 1A, 1D, and 1G provide a side-by-side comparison of MM and iECM. FIGS. 1B, 1E and 1H show the MM solution, and FIGS. 1C, 1F, and 1I show the iECM solution. MM gels after 1 hour, whereas iECM does not gel even after 24 hours.

FIGS. 2A and 2B are images showing a decrease in fluorescent bovine serum albumin (BSA) signal in the heart following infusions of infusible ECM (iECM), relative to saline infused hearts, in a myocardial infarction model. BSA was delivered 30 minutes after iECM infusion. FIG. 2A shows gross images of short axis sections of the hearts. FIG. 2B shows corresponding fluorescent scans of the hearts of FIG. 2A. FIG. 2C is a chart showing quantification. FIG. 2D shows quantification of decrease in BSA signal when BSA was delivered 1, 3, and 7 days after iECM infusion.

FIGS. 3A and 3B are conceptual diagrams schematically showing ECM proteins filling in the gaps of a leaky vessel and coating the lumen of a small vessel. FIG. 3A shows a side cross-sectional view of the leaky vessel. FIG. 3B shows a transverse cross-sectional view of the leaky vessel of FIG. 3A.

FIG. 4A is a brightfield image showing iECM retention in rat lungs during early-stage PAH. FIG. 4B is a corresponding fluorescent image of the rat lungs of FIG. 4A.

FIG. 5A is a fluorescent image showing iECM retention in two rat lungs during late-stage PAH. FIG. 5B is a corresponding brightfield image of the two lungs. FIG. 5C is a magnified image of a portion of FIG. 5B. FIG. 5D is a fluorescent image the histological PAH lung section showing iECM signal localized on the lumen of a pulmonary vessel.

FIG. 6A is a brightfield image showing iECM retention following acid aspiration in rat lungs. FIG. 6B shows a corresponding fluorescent image of the rat lungs of FIG. 6A.

FIG. 7 shows brightfield and fluorescent images of heart and lungs exhibiting iECM retention following acid aspiration in mouse lung.

FIG. 8A is a brightfield image showing iECM retention following intratracheal instillation after acid aspiration in rat lungs. FIG. 8B shows a corresponding fluorescent image of the rat lungs of FIG. 8A.

FIG. 9 shows brightfield and fluorescent images showing tagged iECM retention following LPS systemic injury in mouse brain, heart, lungs, spleen, kidney and liver relative to signal from injury alone, injury with trilysine material control, and healthy control with tagged iECM.

FIG. 10 is an image showing a heatmap of tagged iECM retention following LPS systemic injury in mouse brain, heart, lungs, spleen, kidney and liver relative to signal from injury alone, injury with trilysine material control, and healthy control with tagged iECM.

FIG. 11 is a chart showing quantification of fluorescent organ intensity with LPS injury and different injections.

FIG. 12 is an image showing tagged iECM in injured lung tissue compared to injury only, trilysine (Lys-Lys-Lys), or healthy treated control, with a scale bar of 100 μm.

FIG. 13 is a chart showing a comparison of survival assessment in rats treated with saline infusion or with iECM post-monocrotaline delivery.

FIGS. 14A to 14D are images showing matrix dosage and iECM material retention in organs. FIG. 14A is a brightfield image of harvested organs: (left to right) brain, heart, lungs, spleen, kidneys, and liver. FIG. 14B is a fluorescent image showing a LICOR scan of raw fluorescence intensity scan of iECM tagged with Vivotag-S 750 by NHS-ester chemistry. FIG. 14C is an image showing a heatmap of LICOR fluorescence intensity. FIG. 14D is a chart showing relative fluorescence intensity as a function of organ area (*p<0.05).

FIGS. 15A to 15F are charts showing cytokine expression in iECM treated tissue samples relative to saline treated samples. FIG. 15A shows cytokine expression in the spleen. FIG. 15B shows cytokine expression in the heart. FIG. 15C shows cytokine expression in the kidneys. FIG. 15D shows cytokine expression in the lungs. FIG. 15E shows cytokine expression in the brain. FIG. 15F shows cytokine expression in plasma.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the present disclosure described herein include “consisting” and/or “consisting essentially of” aspects and embodiments.

It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

### Blocking the Leaky Vasculature

Leaky vasculature has been observed in the vessels surrounding tumors and following ischemic injuries, such as myocardial infarction and stroke, and traumatic injury, such as traumatic brain injury. Following ischemic injury reperfusion, inflammatory cytokines and reactive oxygen species enter the tissue to induce cell death and negative tissue remodeling.

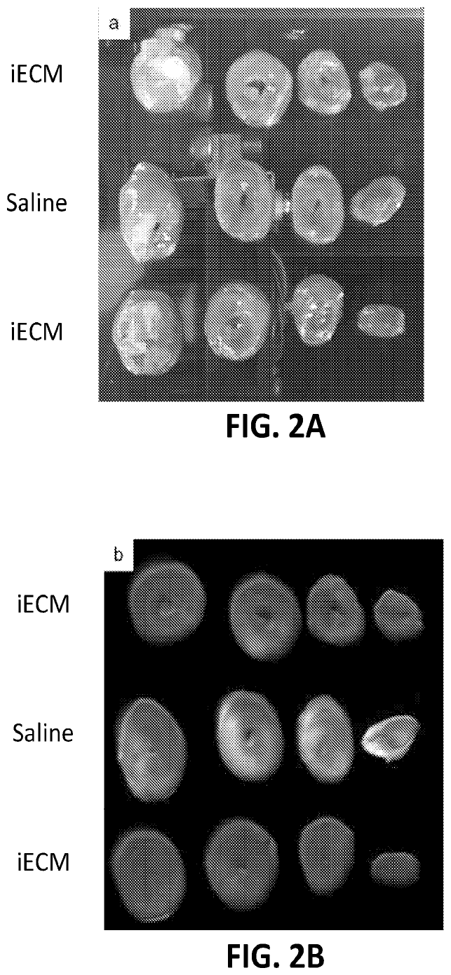

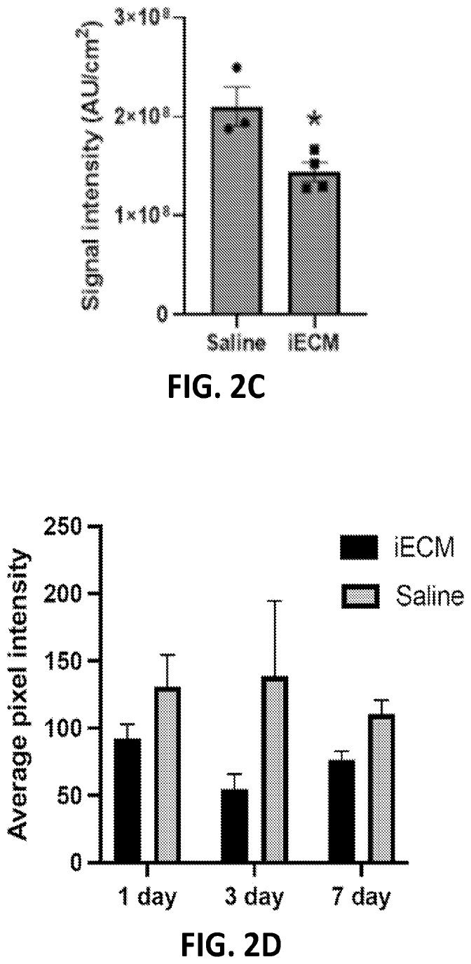

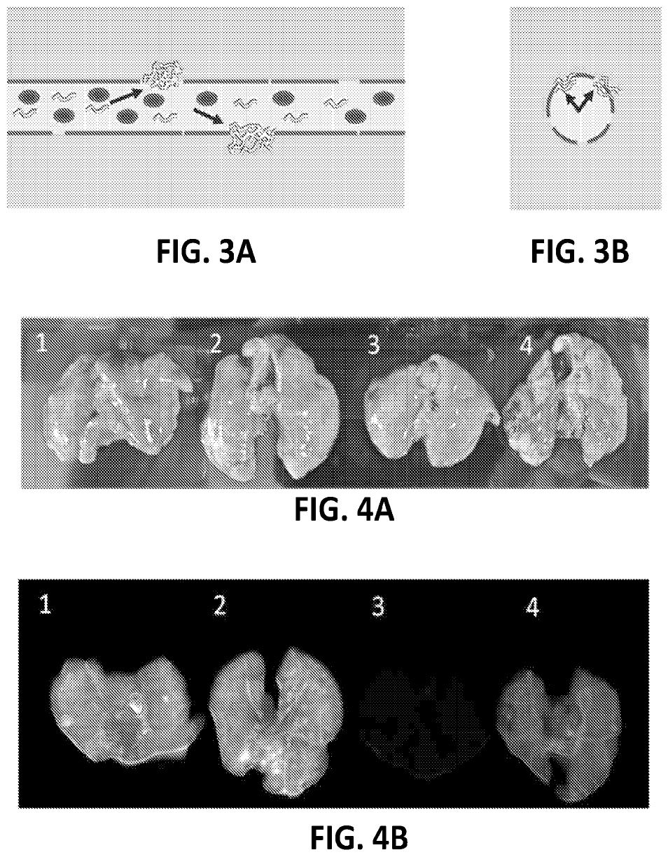

International publication PCT/US2019/058052, which is incorporated by reference herein in its entirety, describes that following ischemic injury and matrix infusions, soluble matrix was observed to coat the lumen of small vessels (capillaries/endothelial cells). The matrix did not block the lumen. Additionally, soluble matrix was shown overlapping endothelial cells in a large vessel, while not blocking the lumen. The present invention, however, shows that infusible ECM can block or fill in the gaps of the leaky vasculature and provide therapeutic benefit by reducing vascular permeability, or preventing harmful molecules and/or inflammatory cells from entering the tissue. The examples describe a myocardial infarction model, and show decreased signal of intravenously infused fluorescent bovine serum albumin (BSA) into the infarct after infusible matrix infusion, suggesting that infusible matrix prevented the fluorescent BSA from entering the tissue, and, therefore, reduced tissue permeability (FIGS. 2A, 2B, and 2C). Additionally, the infusible ECM reduced the amount of fluorescent BSA entering the tissue out to one week after ECM delivery (FIG. 2D). Since the infusible ECM degrades by 3 days, this shows that it also accelerates vascular healing and closure. A schematic in FIGS. 3A and 3B shows infusible matrix coating the lumen of a vessels and filling in the endothelial cell gaps of the leaky vasculature. The use of infusible matrix can be expanded to other conditions involving leaky vessels or disrupted endothelial cell junctions or can be used generally to reduce vascular permeability and accelerate vascular healing.

### Pulmonary Arterial Hypertension

Pulmonary Arterial Hypertension (PAH) is a chronic, progressive disease in which elevated vasculature pressure in the lungs causes significant damage to the lungs, as well as maladaption of the heart, leading to heart failure and death.¹ In patients, pre-disposed to PAH, injury to the blood vessel of the lung results in inflammation that does not resolve. Apoptosis of the endothelial cells that line the pulmonary vessels give rise to apoptotic-resistant endothelial cells, which continue to proliferate in spite of contact inhibition.^{2 3} This reduces the area of the vessel lumen, increasing the blood pressures and further damages the blood vessels, causing more EC proliferation and higher pressures. The heart compensates for the increased pulmonary pressures by increasing the right ventricle (RV) volume and RV free wall thickness up to a point. Then, while the RV volume continues to increase, the RV free wall begins to thin and become fibrotic. This eventually leads to heart failure.¹

An infusible extracellular matrix (ECM) composition can be used in therapy to prevent inflammation damage and apoptosis in endothelial cells lining blood vessels within the lungs. As described above, the infusible ECM binds to leaky or damaged vessels of the heart and may protect them from damage caused by oxidative stress following MI. The same properties may prevent the progression of PAH by protecting endothelial and smooth muscle cells that make up blood vessels, thereby preventing increased inflammation and further damage. In the same vein, patients with advanced PAH shown maladaption of the right ventricle free wall of the heart, including muscle thinning, high levels of inflammation and fibrosis and loss of vasculature. iECM coating of heart vessels may reduce or prevent chronic inflammation and further loss of vasculature within the RV free wall.

iECM exhibits increased retention, 24 hours after delivery, in the lungs of pulmonary hypertensive rats during early and late phases of monocrotaline-induced pulmonary hypertension. The infusible matrix may bind to the lumen of injured lung vessels, but not in the vessels of healthy lungs.

### Acute Respiratory Distress Syndrome

Acute Respiratory Distress Syndrome (ARDS) is a critical illness defined by low blood oxygen levels and multifocal airspace disease on chest imaging as the result of an insult known to cause injury to the lungs.⁴ ARDS, often referred to as Acute Lung Injury (ALI) in animal models, may be due to insults that cause direct injury to the alveolar epithelium (i.e. inhalation injury) or those that cause indirect injury via the pulmonary vascular endothelium (i.e. sepsis).^{4,5} In all cases of ARDS, inflammation associated with damage to the alveolar-capillary membrane allows for the accumulation of fluid within the alveolar space and impaired gas exchange. Multiple ARDS animal models of direct and/or indirect lung injury have been established to test the therapeutic abilities of potential treatments. For example, instillation of hydrochloric acid into the lungs is often used to mimic direct lung injury, and systemic injection of lipopolysaccharide (LPS) is often used to mimic indirect lung injury due to sepsis. The leading cause of death due to ARDS is multiple organ failure due to systemic inflammation and microvascular leak. Due to the properties of iECM to bind to damaged vasculature following intravenous infusion, compositions including iECM may be used to mitigate both the local and systemic consequences of ARDS by preventing leak within the alveolar-capillary membrane as well as remote organs, such as, the kidney.

### Ventilator Induced Lung Injury

Ventilator induced lung injury (VILI) refers to damage to the alveolar capillary membrane due to alveolar overdistention from high driving pressures (barotrauma or volumtrauma) or the repetitive opening and collapse of non-compliant alveoli (atelectrauma).⁶ VILI contributes to a “second hit” of systemic inflammation in critically ill patients who require mechanical ventilation as a life-saving intervention. Open lung protective ventilation with low tidal volumes aimed at preventing VILI is the standard of care in patients who are high risk for ARDS.⁷ Despite this treatment strategy, some patients still develop VILI, and no pharmacotherapies aimed at preventing VILI currently exist. Animal models of VILI most often combine an inflammatory insult, such as LPS, with injurious mechanical ventilation to mimic the two-hit model of systemic inflammation that is observed in many human subjects undergoing mechanical ventilation. These models have demonstrated that lung and remote organ failures due to VILI are characterized by endothelial activation⁶ and microvascular leak.⁸ Therefore, ECM infusion in patients with critical illness who require mechanical ventilation would be predicted to improve mortality by preventing VILI and subsequent remote organ failures. Administration of ECM at the onset of mechanical ventilation would also offer the opportunity to utilize ECM as a preventative therapy for VILI.

### Systemic Inflammation/Cytokine Storm/Sepsis/Systemic Viral Infections

Implantable or injectable material scaffolds are limited in the deployment and treatment within a single diseased tissue, however, applications for systemic responses from a localized tissue disease, comorbidities, or systemic disease conditions are more limited. Conditions such as sepsis or systemic viral infections such as COVID-19 leading to dysregulated systemic inflammation or cytokine storm can lead to damage not only in the lungs, but also amongst other organs such as the heart,⁹ brain,^{10,11} or the kidneys. Methods to treat systemic inflammatory conditions such as targeting specific cytokines or immune cell populations are limited as the response is driven by a large host of different inflammatory pathways. Decellularized biomaterials such as myocardial matrix have previously demonstrated immunomodulation of the inflammatory response across multiple immune cell populations and stimulation of endogenous remodeling outcomes supporting tissue repair.^{12,13} Furthermore, this immunomodulatory effect has also been observed with soluble fractions of these decellularized materials¹⁴ and mitigation of acute localized cardiac tissue damage with retention observed in other organs.¹⁵ In an extension of this potential to circulate and extravasate from leaky vasculature or the enhanced permeability and retention effect into multiple tissues that occurs with endothelial cell disruption, favorable size and hemodynamic properties for colloid and infusible ECM materials have the potential for systemic application for treating damage and inflammation across multiple organs.¹⁵⁻¹⁷

The present disclosure provides infusible ECM compositions and methods for treatment of pulmonary diseases, such as chronic obstructive pulmonary disease, asthma, pulmonary artery hypertension. The infusible ECM compositions can be infused to treat injured tissues and/or endothelial cells of the lungs.

The present disclosure provides infusible ECM compositions and methods for treatment of Ventilator Induced Lung Injury (VILI) and VILI associated with stomach content aspiration. Multi-factorial injury associated with VILI includes mechanical injury to the alveoli and surrounding microvasculature, protein denaturation due to low pH, and associated inflammation due to the injuries described above, as well as inflammation damage due to stomach content aspiration and bacterial infection. The infusible ECM compositions can be infused concurrently or after mechanical ventilation to coat and protect endothelial cells lining the microvasculature against inflammation and other injury. The infusible ECM compositions can be aspirated (by nebulizer) to protect alveoli against mechanical injury, low pH and systemic inflammation or cytokine storm arising from post-infection complications such as with sepsis or viral infections such as COVID-19.

The present disclosure provides infusible ECM compositions and methods for the treatment of tissue damage and inflammation associated with the inhalation of environmental toxins. The infusible ECM compositions delivered by intravenous infusion or aspiration/inhalation will coat damaged endothelium or alveolar epithelium to suppress leukocyte and inflammatory cytokine infiltration as well as protect damaged tissues from further toxin exposure (i.e. toxic molds, airborne environmental or industrial pollutants).

The present disclosure provides a non-invasive biomaterial therapeutic composition including infusible ECM, for example, cardiac infusible ECM, that can adhere to sites of vascular leak in systemic inflammation. The composition provides dampened inflammatory response in multiple tissues, for example, in multi-organ inflammation in the lungs, heart, brain, kidneys, spleen, and blood. The composition may significantly reduce markers of endothelial dysfunction while also upregulating genes related to alternative immune cell activation. Without being bound by theory, systemically circulating infusible ECM can provide immunomodulatory effects against excess systemic inflammation, thus mitigating multi-organ tissue damage induced by infectious disease.

For human therapy, there are many source species for the extracellular matrix: e.g., human, porcine, bovine, goat, mouse, rat, rabbit, chicken, and other animal sources. Furthermore, there are many tissue sources: e.g., heart, brain, bladder, small intestine, skeletal muscle, kidney, liver, lung, blood vessels and other tissues and organs.

In embodiments, the tissue is first decellularized, leaving only the extracellular matrix such as disclosed in U.S. Patent Publication US2013/0251687, for example, which is incorporated by reference in its entirety. The matrix is then lyophilized, ground or pulverized into a fine powder, solubilized with pepsin or other enzymes, and subsequently neutralized and buffered as previously reported. Following neutralization, the digestion (pre-gel solution) is fractionated to separate soluble and insoluble fractions. Processing the separation of soluble and insoluble fractions may be achieved by centrifugation, dialysis, filtration, or adjusting pH or salinity. The soluble fraction can be dialyzed to remove salts, lyophilized, and resuspended to adjust ECM concentration. ECM can be sterile filtered, lyophilized, and stored in sterile containers. ECM can be resuspended to appropriate/physiological concentration for infusion.

An infusible ECM composition refers to extracellular matrix material which has been decellularized, lyophilized, ground, and digested and having at least a portion of the solid components removed therefrom. In embodiments, an infusible ECM composition is obtained from centrifugation supernatant. In embodiments, infusible ECM composition is able to pass through a filter size of less than 1 μm, 500 nm, 250 nm, 220 nm, or 200 nm. The infusible ECM composition having at least a portion of solid ECM components with which it naturally occurs removed therefrom is a more transparent material than before removal of the ECM solids. However, it is to be understood that some degree of insoluble small particulate matter, such as ECM colloids, ECM nanofibers, or ECM nanoparticles may still be present in the infusible ECM composition. An infusible ECM composition has been substantially isolated when at least 50%, 60%, 70%, 80%, 90%, 95%, 98% or 99% of the naturally occurring ECM solids by volume have been removed therefrom.

After adjusting concentration and/or sterile filtration, the infusible ECM composition can be lyophilized and stored frozen (e.g. −20 C, −80 C) for at least 3 months. The infusible ECM composition can then be rehydrated with sterile water prior to injection or infusion.

The infusible ECM composition can be infused through a catheter, delivered intravenously, or by intravascular infusion with or without a balloon. The infusible ECM composition can pass through healthy vascular and then bind to damaged leaky vasculature, such as that found in an acute myocardial infarction, stroke, other ischemic tissues, tumors, etc.

The infusible ECM composition gel can be crosslinked with glutaraldehye, formaldehyde, bis-NHS molecules, or other crosslinkers. The infusible ECM composition can be combined with cells, peptides, proteins, DNA, drugs, nutrients, survival promoting additives, proteoglycans, and/or glycosaminolycans. The infusible ECM composition can be combined and/or crosslinked with a synthetic polymer. The infusible ECM composition can be used alone or in combination with above described components for endogenous cell ingrowth, angiogenesis, and regeneration. The infusible ECM composition can be use alone or in combination with above described components as a matrix to change mechanical properties of the tissue. The infusible ECM composition can be delivered with cells alone or in combination with above described components for regenerating damaged tissue.

In embodiments, the present disclosure provides compositions and methods for treatment of vascular injury, leaky vasculature, including but not limited to reducing or blocking the leaky vasculature permeability, reducing the infiltration of immune cells, exudate, reactive oxygen species, inflammatory cytokines, growth factors, exosomes, or any proteins, particles, or molecules that may be deleterious to tissue function, result in negative tissue remodeling, or disease progression. Infusible matrix infusions could be used as treatments for injuries involving leaky vasculature, including tumors, myocardial infarction, stroke, and other ischemic conditions.

In embodiments, the present disclosure provides that the infusible ECM is combined with cells, peptides, proteins, DNA, drugs, nanoparticles, antibiotics, growth factors, nutrients, exosomes and extracellular vesicles, survival promoting additives, proteoglycans, and/or glycosaminoglycans.

In embodiments, the present disclosure provides that the composition of infusible extracellular matrix is derived from human, animal, embryonic, and/or fetal tissue sources. In embodiments, the present disclosure provides that the composition of infusible extracellular matrix is derived from heart, brain, bladder, small intestine, skeletal muscle, kidney, liver, lung, bronchioles, blood vessels, and other tissues/organs tissue sources.

In embodiments, the present disclosure provides a method for treating pulmonary arterial hypertension (PAH) comprising infusing in a subject in need with PAH an effective amount of a composition comprising infusible decellularized extracellular matrix derived from muscle, lung tissue or other tissues.

In embodiments, the present disclosure provides that the infusible ECM composition does not gel in vitro or inside the blood in vivo, but does gel when administered in tissue in vivo.

In embodiments, the present disclosure provides that said composition is delivered intravenously by infusion. In embodiments, the present disclosure provides that said composition is delivered by intracoronary infusion with a balloon infusion catheter. In embodiments, the present disclosure provides that said composition transitions to a gel form in tissue after delivery. In embodiments, the present disclosure provides that said composition transitions to form a coating on the endothelium of injured blood vessels after delivery. In embodiments, the present disclosure provides that said composition degrades within one to 14 days following injection or infusion.

In embodiments, the present disclosure provides that the injection or infusion of said composition repairs damage to the lungs or cardiac muscle sustained by said subject, such as a right ventricle heart failure, associated with PAH. In embodiments, the present disclosure provides that said effective amount is an amount that increases blood flow, increases viable tissue mass, or induces new vascular formation in the area of the injection or infusion of the subject. In embodiments, the present disclosure provides that said effective amount is an amount that promotes cell survival, reduces inflammation, accelerates vascular healing, and repairs damaged vasculature in the area of the injection or infusion of the subject.

In embodiments, the present disclosure provides protection against acute respiratory distress syndrome (ARDS) with or without ventilator induced lung injury (VILI). In embodiments, the present disclosure provides delivery of infusible ECM via intravenous infusion, lung lavage or aspiration by nebulization for the protection of alveolar epithelium as well as endothelial cells and other microvascular and vascular components within the lung, heart and peripheral organs and tissues. In embodiments, the present disclosure provides delivery of infusible material before, concurrently, or after tissue damage associated with ventilator injury, stomach content aspiration, or accidental or deliberate inspiration of environmental toxins.

In embodiments, the present disclosure provides methods for treatment of injury associated with lung damage, cardiac damage or multi-organ tissue damage occurring from systemic inflammation/cytokine storm or occurring from post-infection complications from a bacterial or viral source such as COVID-19, or an associated tissue disease comorbidity. In embodiments, the present disclosure provides methods for treatment of injury associated with COVID-19 within injured lungs when delivered intravenously or via intratracheal instillation.

In embodiments, when delivered to a patient in an effective amount, iECM reduces vascular permeability by affecting vascular healing after damage or by physical blockage in damaged areas or both. In embodiments, when delivered to a patient in an effective amount, iECM improves vascular healing, vascular repair, or healthier blood vessels. In embodiments, an effective amount of iECM is delivered to the cardiac vasculature to treat conditions related to a myocardial infarction. In embodiments, an effective amount of iECM is delivered to the systemic vasculature to treat conditions related to sepsis or a viral infection. In embodiments, an effective amount of iECM is delivered to lung vasculature to treat pulmonary arterial hypertension. In embodiments, an effective amount of iECM is delivered to lung vasculature to treat pulmonary arterial hypertension, acute respiratory distress syndrome (ARDS), or ventilator injury.

In embodiments, the iECM may include soluble ECM or transparent colloid ECM. In embodiments, the iECM includes ECM-derived nanofibers, nanorods, or nanoparticles less than 0.22 micrometers. In embodiments, the iECM is a shear thinning liquid. In embodiments, a soluble solution includes an effective amount of iECM. In embodiments, an iECM composition includes a colloid composition or a transparent colloid solution. In embodiments, an iECM composition includes one or more of nanofibers, nanorods, or nanoparticulates or a combination of any of nanofibers, nanorods, or nanoparticulates. In embodiments, an iECM composition includes nanofibers less than 0.40 micrometers in diameter. In embodiments, an iECM composition includes nanofibers less than 0.35, 0.30, or 0.25 micrometers in diameter. In some such embodiments, the iECM composition includes nanofibers less than 0.22 micrometers in diameter. In embodiments, an iECM composition includes nanofibers between 0.1-0.40 micrometers in diameter. In embodiments, an iECM composition includes nanofibers between 0.05-0.30 micrometers in diameter. In embodiments, an iECM composition includes nanofibers between 0.10-0.25 micrometers in diameter.

In embodiments, an iECM composition includes a material that does not gel in vitro when exposed to temperatures at or below 37° C. In embodiments, an iECM composition includes a material that does not gel in vitro when exposed to temperatures at or below 39° C. In embodiments, an iECM composition includes a material that does not gel in vitro when exposed to temperatures at or below 42° C. In embodiments, an iECM composition includes a material that does not gel in vivo unless delivered into a solid tissue. For example, the iECM composition may include a material that does not gel in vitro when exposed to temperatures at or below 37° C.

In embodiments, an iECM composition includes a material that does not gel in vitro or in vivo when delivered in the blood or systemic circulation. In embodiments, an iECM composition includes a material that does gel in vivo when delivered into a solid tissue. In embodiments, an iECM composition includes a material that does gel in vivo when exposed to vascular tissue. In embodiments, an iECM composition includes a material that does not gel in vitro or in vivo when delivered into blood, but does gel in vivo when exposed to tissue, or exposed to vascular tissue.

In embodiments, without being bound by theory, iECM is able to be delivered through a catheter or needle for treatment of a malady because iECM is shear-thinning. In embodiments, the catheter or needle can be up to 3 to 5 m in length and have an diameter of 30 g or greater.

In embodiments, an effective amount of iECM delivered to a patient includes a concentration of ECM about 1-20 mg ECM to mL of total product. In embodiments, an effective amount of iECM delivered to a patient includes a concentration of ECM about 2-10 mg ECM to mL of total product. In embodiments, an effective amount of iECM delivered to a patient includes a concentration of ECM about 3-6 mg ECM to mL of total product. In embodiments, an effective amount of iECM delivered to a patient includes a concentration of ECM about 4, 5, or 6 mg ECM to mL of total product.

In embodiments, iECM is delivered in an effective amount to a patient once, or is delivered to a patient multiple times. In embodiments, iECM is delivered to a patient on a schedule or in multiple doses, for example, once a day, once a week, once a month, once a year or more or less frequently.

In embodiments, iECM is delivered at different time courses throughout a patient's disease as is most appropriate, for example, immediately post-injury, infection, or diagnosis, or additionally, about one hour, several hours, one day, one week, one or more months, or one or more years after injury, infection, or diagnosis.

In embodiments, iECM is delivered following an angioplasty. In embodiments, iECM is delivered after a patient is placed on a ventilator. In embodiments, iECM is delivered when a patient is admitted to the ICU. In embodiments, iECM is delivered to a patient with pulmonary arterial hypertension prior to right ventricular heart failure. In embodiments, iECM is delivered to a patient with pulmonary arterial hypertension after onset of right ventricular heart failure.

In embodiments, iECM reduces vascular permeability by including, consisting of, or consisting essentially of an effective amount of nanosized particles or nanofibers less than 0.22 micrometers in diameter. In embodiments, iECM reduces vascular permeability by including, consisting of, or consisting essentially of an effective amount of nanosized particles or nanofibers less than 0.22 micrometers in diameter that offer a biologic response within the tissue that promotes vascular healing.

In embodiments, iECM reduces vascular permeability by binding to the exposed ECM in the vasculature through peptides, proteins, or polysaccharides in the iECM. In embodiments, iECM reduces vascular permeability by binding to the exposed basal lamina in the vasculature through peptides, proteins, or polysaccharides in the iECM. In embodiments, iECM reduces vascular permeability by binding exposed receptors on endothelial cells including integrins, selectins, and/or other transmembrane receptors through peptides, proteins, or polysaccharides in the iECM.

In embodiments, iECM reduces vascular permeability by binding to vasculature in areas of low shear stress (shear rate <1000, <500, <200, <100 s⁻¹). In embodiments, iECM accelerates vascular healing by attaching to leaky endothelium and then binding platelets in areas of low shear stress (shear rate <1000, <500, <200, <100 s⁻¹). In embodiments, iECM accelerates vascular healing by attaching to leaky endothelium and then attracting endothelial or vascular progenitor cells in areas of low shear stress (shear rate <1000, <500, <200, <100 s⁻¹).

### EXAMPLES

### Example 1: Infusible ECM does not Gel at 37 Degrees Celsius

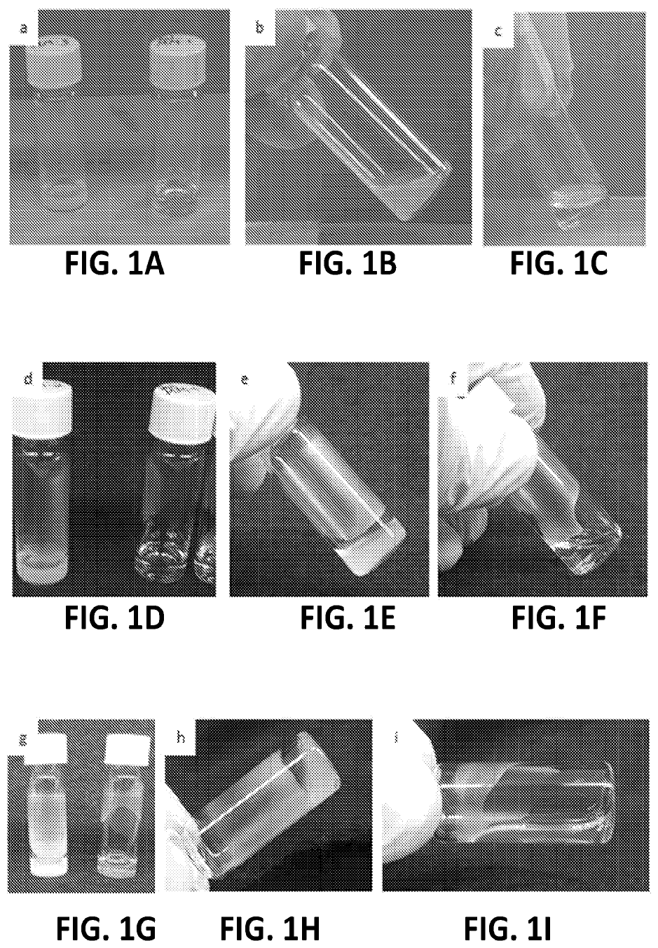

Lyophilized myocardial matrix (MM) and infusible ECM (iECM) were resuspended to 6 mg/ml and 10 mg/ml, respectively, and 500 μL of each were dispensed into scintillation vials (FIG. 1A). Both MM and iECM were liquid and flowed (FIGS. 1B and 1C). Vials were transferred to a 37 degree Celsius incubator. After 1 hour of incubation, MM and iECM were imaged (FIG. 1D). MM formed a gel (FIG. 1E), whereas iECM was still a liquid (FIG. 1F). Following 24 hours of incubation, MM and iECM were imaged again (FIG. 1G). The MM was still a gel and did not flow (FIG. 1H), whereas iECM was still a liquid and flowed (FIG. 1I).

### Example 2: Using Infusible Extracellular Matrix to Block Gaps Formed in the Leaky Vasculature, and Accelerate Vascular Healing

Leaky vessels were induced in an ischemia-reperfusion rat myocardial infarction model, and infusible extracellular matrix was infused to the coronary arteries using a clamp intracoronary injection. Fluorescent bovine serum albumin (BSA) was then intravenously infused via a tail vein injection 30 minutes later, and hearts were harvested and scanned for fluorescent signal (FIGS. 2A, 2B, and 2C). In other animals, BSA was delivered 1, 3, and 7 days after iECM infusion, and hearts were harvested and scanned for fluorescent signal (FIG. 2C).

### Example 3: Infusible ECM Retention is Increased in PAH Rats Compared to Healthy Rats

Male Sprague Dawley rats treated with monocrotaline (60 mg/kg) showed increased retention of fluorescently-labelled iECM (10 mg/mL, Vivotag 750), 24 hours after tail vein infusion in early and late PAH. Histological sections of PAH rat lung show binding of iECM to the endothelial cells lining the vascular lumen.

FIGS. 4A and 4B show brightfield and fluorescent images of PAH (2 weeks post MCT delivery) and healthy lungs, 24 hours after fluorescently-labelled iECM. 1&2) Lungs from PAH rats that received dyed iECM. 3) Lungs from a PAH rat that received PBS control. 4) Healthy rat that received dyed iECM.

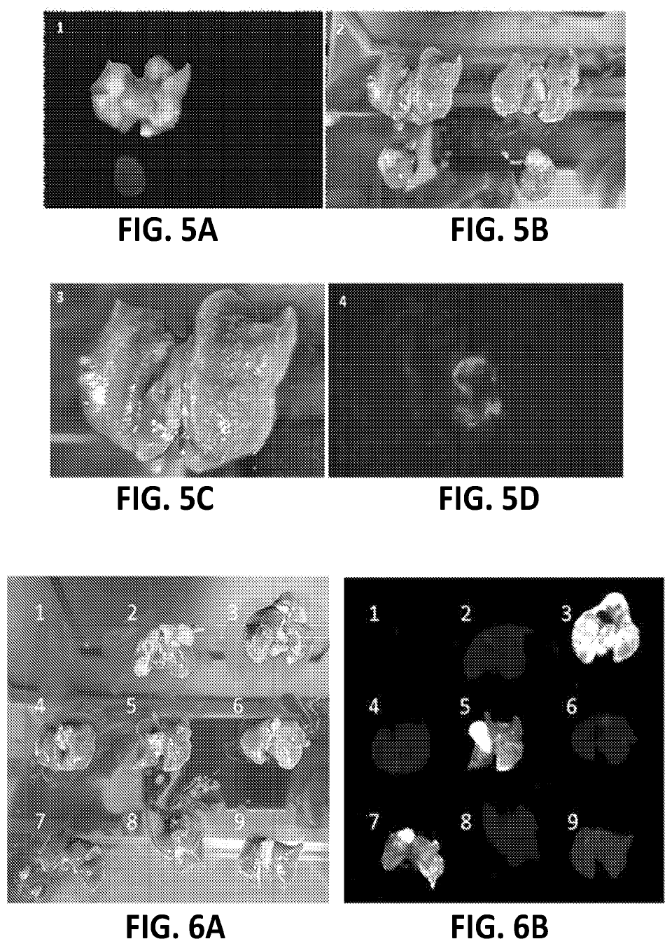

FIGS. 5A, 5B, 5C, and 5D show brightfield and fluorescent images of PAH (5 weeks post MCT delivery). FIG. 5A is a fluorescent image showing iECM retention in two rat lungs during late-stage PAH. FIG. 5B is a corresponding brightfield image of the two lungs. The left lung received dyed iECM, the right lung received PBS. FIG. 5C is a magnified image of a portion of FIG. 5B. FIG. 5D is a fluorescent image the histological PAH lung section showing iECM signal localized on the lumen of a pulmonary vessel.

### Example 4: Infusible ECM Retention is Increased with Increasing Hydrochloric Acid Concentration Following Acid Aspiration in Rat Lungs

Sprague-Dawley rats received lung aspiration of HCl and then received 250 uL of fluorescently-dyed iECM (10 mg/mL, Vivotag750) or PBS control. Results show increased iECM signal in rat lungs that received increasing concentration of HCl. Healthy lungs showed very little retention.

FIGS. 6A and 6B shows brightfield and fluorescent images of rat lungs that received 150 uL of HCl by aspiration, or were healthy, and then received 250 uL of fluorescently-dyed iECM (10 mg/mL, Vivotag750) or PBS control, by tail vein injection. Lungs were harvested and imaged 24 hours later. Animal treatments: 1) Blank. 2) 0.5M HCl, PBS. 3) 0.25M HCl, iECM. 4) 0.25M HCl, PBS. 5) 0.1M HCl, iECM. 6) 0.1M HCl, PBS. 7) 0.05M HCl, iECM. 8) 0.05M HCl, PBS. 9) Healthy, iECM

### Example 5: iECM Retention in Mouse Lungs Following Acid Aspiration

C57/B6 mice received lung aspiration of 50 uL of 0.2M HCl or nothing (healthy) and then one hour later received fluorescently-stained (Vivotag750) iECM (10 mg/mL) via tail vein injection. 24 hours later the heart and lungs were removed and imaged by brightfield and fluorescent microscopy.

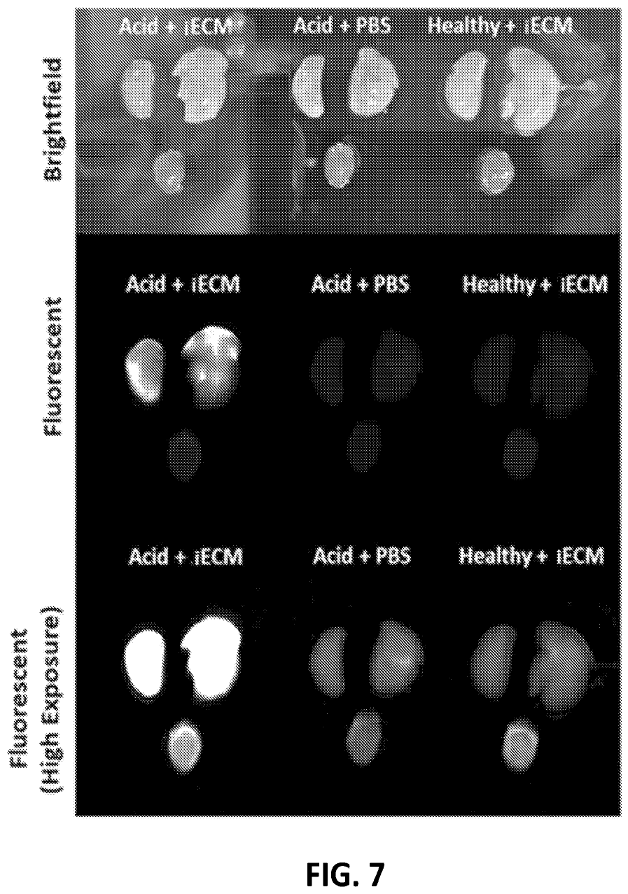

FIG. 7 shows brightfield and fluorescent images of heart and lungs from two mice that received acid aspiration (left and center organs) and a mouse that received no injury (right). The organ sets on the left and right came from mice that received stained iECM and the organ set in the middle receive PBS. The bottom panel shows a fluorescent image with extended exposure time to demonstrate heart fluorescent signal and indicate the presence of all three organ sets not visible in the middle panel.

### Example 6: iECM Retention in Rat Lungs Following Intratracheal Instillation after Acid Aspiration

Sprague-Dawley rats received lung aspiration of HCl and then received intratracheal administration of fluorescently-stained (Vivotag750) iECM (10 mg/mL). Results show iECM signal in the lungs.

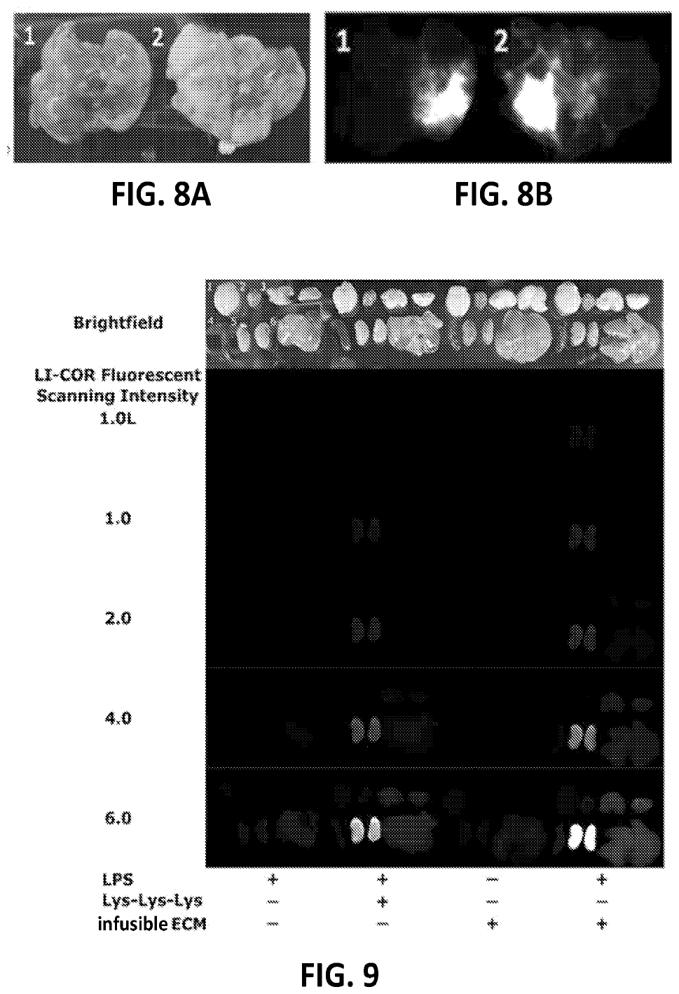

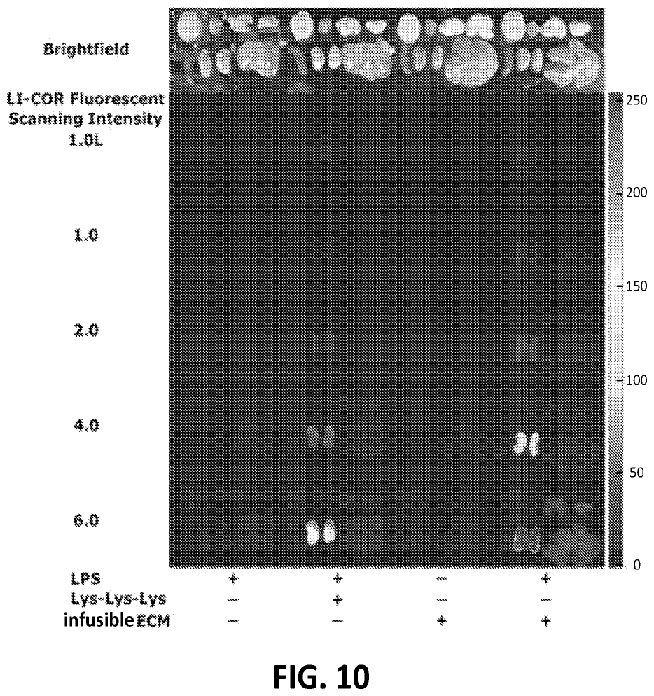

FIGS. 8A and 8B show brightfield and fluorescent images of lungs from two rats that received acid aspiration (left and center organs) followed by intratracheal instillation of iECM.

### Example 7: iECM Retention in Mouse Lungs Following LPS Induced Inflammation

FIG. 9 shows brightfield and LICOR Odyssey scanned fluorescent images of 1) brain, 2) heart, 3) lungs, 4) spleen, 5) kidneys, and 6) liver from four mice that received (left to right) intraperitoneal injection of LPS with tail vein injection of saline, intraperitoneal injection of LPS with tail vein injection of 10.4 mg/kg trilysine (Lys-Lys-Lys), tail vein injection of 60 mg/kg of iECM, and intraperitoneal injection of LPS with tail vein injection of 60 mg/kg of iECM. LPS administration was done 4 hours preceding tail vein injection, and organs were perfused with 30 mL of 1×PBS and harvested at 20 hours post-tail vein injection. Trilysine and iECM were tagged with fluorescent probe Vivotag750 by NHS-ester chemistry.

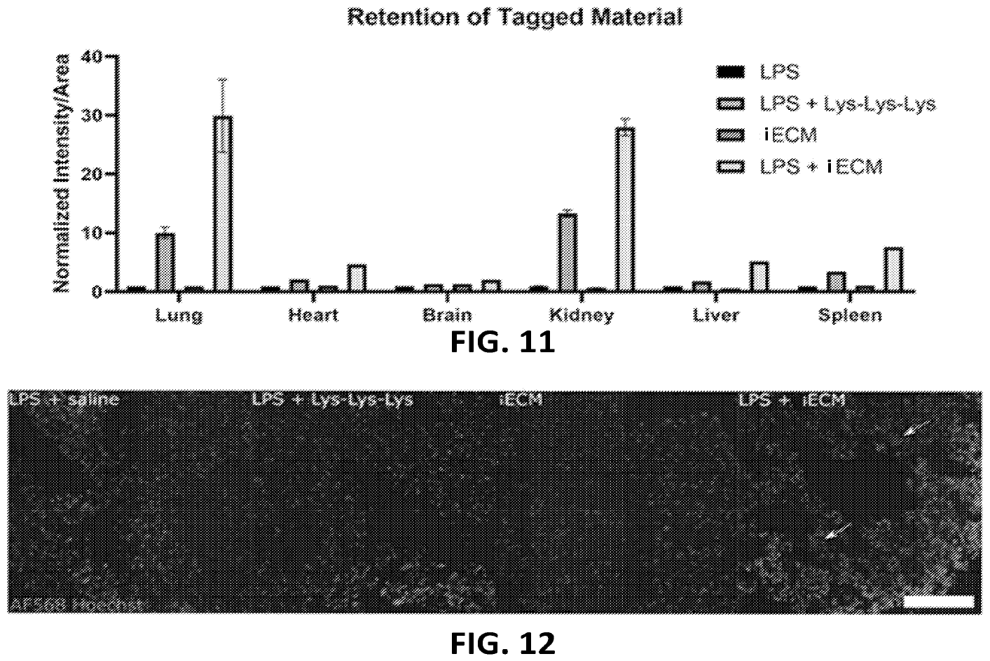

FIG. 11 shows quantification of fluorescent signal from LICOR Odyssey scanned images of brain, heart, lungs, spleen, kidneys, and liver from LPS induced systemic inflammation tissue injury only control, LPS induced systemic inflammation tissue injury injected with trilysine control tagged with Vivotag750, healthy mouse intravenously injected with iECM tagged with Vivotag750, and LPS induced systemic inflammation tissue injury injected iECM tagged with Vivotag750.

FIG. 12 shows representative fluorescent images from fresh frozen mouse lung tissue that received (left to right) intraperitoneal injection of LPS with tail vein injection of saline, intraperitoneal injection of LPS with tail vein injection of 10.4 mg/kg trilysine (Lys-Lys-Lys), tail vein injection of 60 mg/kg of iECM, and intraperitoneal injection of LPS with tail vein injection of 60 mg/kg of iECM. Trilysine and iECM were tagged with fluorescent probe Alexa Fluor 568 by NHS-ester chemistry. Counterstain of Hoechst 33342 was used to stain nuclei. Images were scanned on a Leica Ariol® DM6000B system. Scale bar of 100 μm.

### Example 8: Pulmonary Arterial Hypertension (PAH) Model

The survival assessment following double dose infusion of heart-derived infusible ECM (iECM) or saline control was assessed. Male Sprague Dawley rats received 60 mg/kg of Monocrotaline (MCT) in sterile saline via subcutaneous injection (1 mL). At two weeks post-MCT delivery, rats were assigned at random to receive either heart-derived iECM (10 mg/mL in sterile water, n=5) infusion or sterile saline infusion (n=5) by tail vein injection (5004). At four weeks post MCT, groups received an additional dose of heart-derived iECM or saline infusion by tail vein injection. The study was continued out to 6 weeks post MCT delivery.

Animals that received a double dose of iECM survived approximately 6 days longer (at 50% survival) than the saline control animal groups showing potential as a therapy for PAH (FIG. 13).

### Example 9: Retention of iECM is Dose-Dependent and Increased Across Multiple Organs with LPS Induced Injury

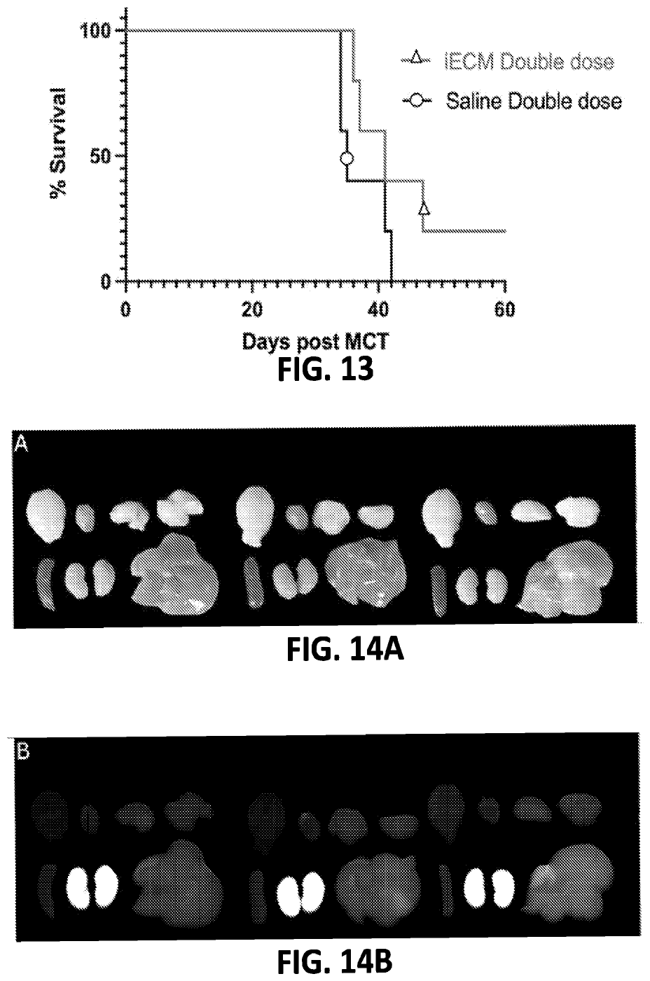

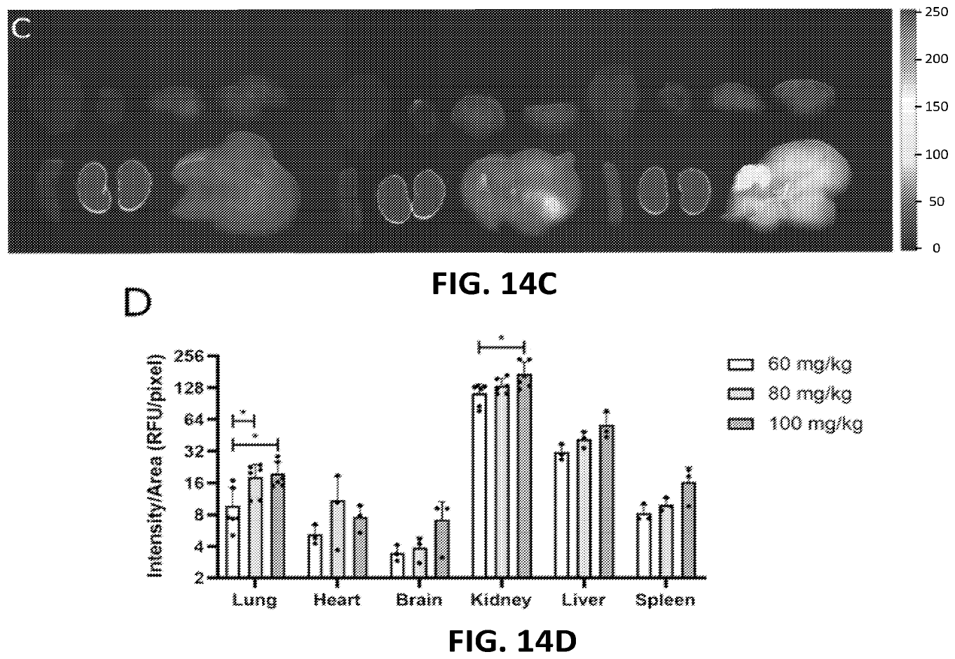

Applicability of iECM for treatment of systemic inflammation was evaluated based on its multi-organ retention and preferential retention in the disease condition. Dosages were evaluated based on greatest average retention of iECM amongst multiple organs (brain, heart, lungs, spleen, kidney, liver) of LPS-induced mice (FIG. 14A). iECM tagged with Vivotag-S 750 (NHS-ester chemistry) was delivered at 60, 80, or 100 mg/kg. A dose of 100 mg/kg was not exceeded as this was the maximum deliverable volumetric dosage for 10 mg/mL iECM at 1/10 mouse blood volume. Fluorescence intensity of organs harvested at 16-24 hours post-iECM injection was measured using LICOR imaging system (FIGS. 14B and 14C). Maximum average retention in the majority of organs was determined at maximum deliverable dose of 100 mg/kg with significant increases for lung and kidneys compared to lowest dose (FIG. 14D). No detrimental or differential responses to different dosages were observed throughout procedures.

Enhanced tissue retention of the iECM material due to disease condition was evaluated by comparing delivery to healthy mouse controls. Additionally, enhanced retention due to material properties of iECM was evaluated in the LPS injury model by comparing to retention of tagged trilysine as a simple peptide-dye conjugate control. The relative primary amine content of iECM to trilysine was determined to control for similar amounts of unreacted free dye molecules and fluorescently tagged peptides between groups. The same set of tissues to dosing assessments (brain, heart, lungs, spleen, kidney, liver) were isolated (FIG. 14A) and fluorescently scanned along with tissue from an untreated healthy mouse to visualize inherent tissue autofluorescence (FIGS. 14B and 14C). iECM retention in the LPS model compared to both retention in healthy mice and trilysine retention in LPS mouse controls was significantly greater for both lungs and kidneys. Trending results were also determined for liver and spleen tissue, while average signal was generally higher for both heart and brain (FIG. 14D).

### Example 10: iECM Treatment of LPS Mice and Initial Screening of Therapeutic Efficacy

After determining dosing parameters and confirming enhanced material retention of iECM with LPS injury, a set of LPS injured mice were treated with iECM or saline to assess mitigation of inflammatory responses with weight and temperature recorded for exclusion criteria. Of the treated mice, a 30% mortality rate was observed amongst mice that received treatment (2 matrix, 5 saline) before euthanasia for sample collection. Efficacy of iECM treatment was assessed from samples by real time quantitative PCR (RT-qPCR) by screening a set of pro-inflammatory associated markers (Ifng, Il1b, & 116). All five organs demonstrated significant or trending reduction in 116 expression, lung and heart showed trending and significant reduction in Il1b, respectively, and no significant differences in Ifng were determined. Overall, these results supported some degree of immunomodulation of the systemic inflammatory response.

### Example 11: iECM Treatment Significantly Modified Only B Cell Presence in Lung Tissues

Single cell suspension isolated from lung and spleen tissues underwent flow assessment. Spleen tissue demonstrated no significant differences in assayed immune cell presence relative to iECM treatment. Lung tissue demonstrated no significant differences other than increased B cell presence (p=0.03) with iECM treatment. This increase could represent an improved humoral immune response in iECM treated animals versus saline treated animals. With no significant changes in T cell presence in lung or spleen, B cell activation could have been T cell independent in this case. Fewer significant changes here could also be linked to high model severity within a short time window.

### Example 12: iECM Treatment Significantly Alters Inflammatory Profile Amongst Multiple Organs

An assessment of the immune profile was conducted by a Nanostring multiplexed gene expression panel. Results determined a host of differentially expressed genes comparing the iECM to saline treated lungs, heart, brain, and kidneys. For the lungs, downregulation of a host of pro-inflammatory associated cytokines (Il1a, Il17a, Il6, Tslp), chemokines (Ccl4, Cxcl1, Cxcl3, Cxcl15), receptors (Tnfrsf9, Tnfsf15), secretory proteins (Tnfaip6), transcriptional regulators (Hif1a), and enzymes (Ptgs2) was observed. Additionally, reduced expression of markers associated with other mechanisms involved in sepsis pathology were also determined such as apoptosis (Tnfrsf8), and endothelial activation and dysfunction (Cd44, Cd80, Vcam1). Several anti-inflammatory (Klrc1, Il10) and regulatory (1115, Socs1, Socs3) markers were also downregulated that were likely compensatory to the overall pro-inflammatory profile. Upregulated genes included anti-inflammatory and regulatory markers (Cmklr1, Cd163, Lif, Ltf), and adaptive immunity cytokines (Il7) and chemokines (Cxcl13). Several pro-inflammatory associated markers such as Il12b were also upregulated. However, given its functional role of enhancing lytic activity T and natural killer cells, upregulation could be considered beneficial to effectively continue combatting an infectious agent.

Similar differential expression patterns were seen in the other organs, which was assessed by comparing consistently differentially expressed genes across multiple organs for evaluating the overall systemic immune response. All four organs showed upregulation of Lair1, a regulatory receptor on cytolytic function of T cells, B cells, and natural killer cells, and downregulation of pro-inflammatory neutrophil chemokine, Cxcl1, and pro-inflammatory cytokine, 116. Other upregulated genes consistent across at least two organs were general or adaptive immune cell development (Btk, Cd48, Il7, Ptpn6), signaling (Btk, Il11ra1), immune surveillance progression (Icam2, Itgb2), regulation of metabolic (Npc1, Pparg) and immune responses (Il17re, Pparg, Sigirr), and pro-inflammatory signaling markers (Traf5). Several pro-inflammatory markers were downregulated including cytokines (Csf2), chemokines (Ccl5, Ccl9, Cxcl3), enzymes (Ptgs2), transcription factors (Cebpb), adhesion molecules (Cd14), and secretory proteins (Tnfaip6). Additionally, T cell chemokines (Cxcl11), markers of endothelial dysfunction (Cd44, Cd80), antiviral (Mx1) and antimicrobial (Hamp) markers, immune cell differentiation (Nt5e), and immunoregulatory markers (Ccl24, Ets1, Lif, Litaf, Socs3) were downregulated.

Overall trends in immune response were evaluated by gene set enrichment to analysis across multiple gene set databases. Analysis demonstrated a general downregulation of a host of pathways related to pro-inflammatory cytokines and inflammatory processes, cell signaling cascades, and cellular metabolic processes.

### Example 13: iECM Treatment Significantly Reduces Inflammatory Cytokine Concentration in Plasma as Well as Lung, Heart, Kidney, and Spleen Tissues

Thirteen inflammatory markers were investigated in multiple tissues to assess sepsis-related multi-organ inflammation in the iECM-treated versus saline-treated mouse tissues. Given the previously demonstrated downregulation of multiple inflammatory markers with iECM treatment, data is shown with annotations surrounding assay level of detection and level of quantification—in numerous cases, iECM treatment would bring cytokine concentration closer to the assays level of quantification.

For tissue homogenates, cytokine expression was normalized to total protein content via BCA assay. Cytokine expression of iECM treated tissues samples was normalized to same tissue saline controls (n=6 per group). One or more samples were below the level of detection (LOD) for a specific analyte, so analyte data was excluded. One or more samples were within the detection range, but below the recommended level of quantification (LOQ) for a specific analyte. Statistical comparisons between iECM and saline treatment were made using unpaired t-tests for each analyte with correction for multiple comparisons using the Holm-Sidak method.

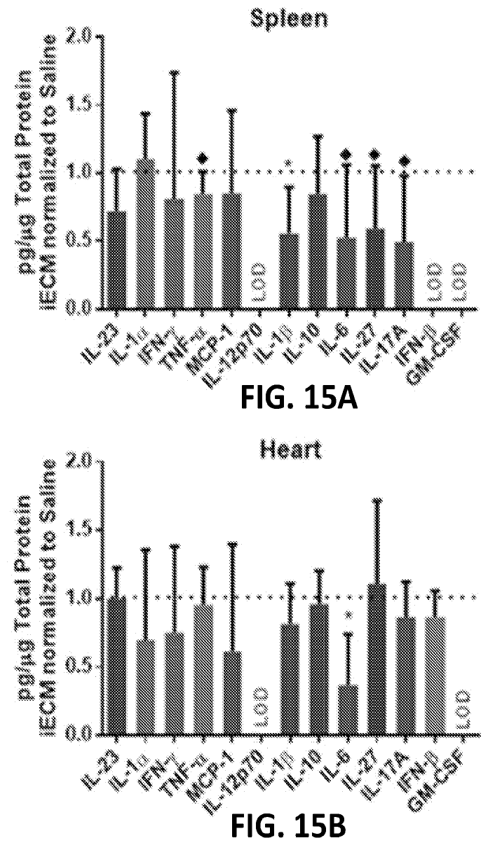

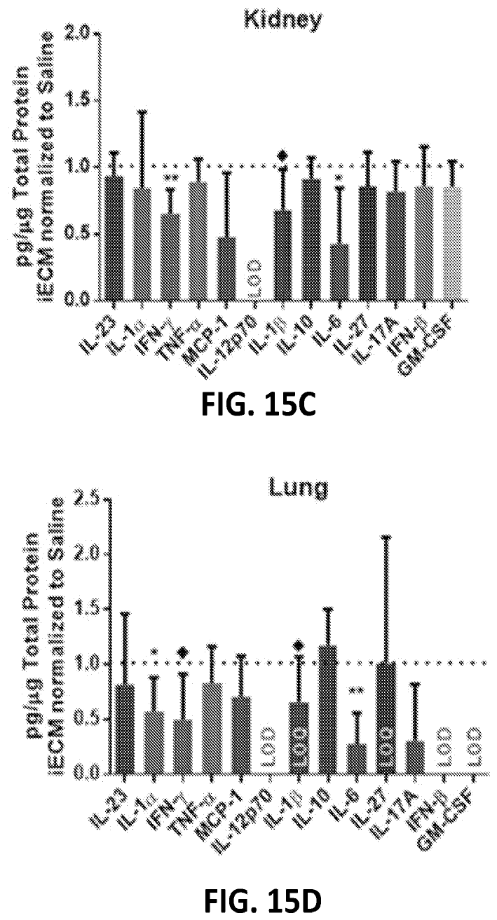

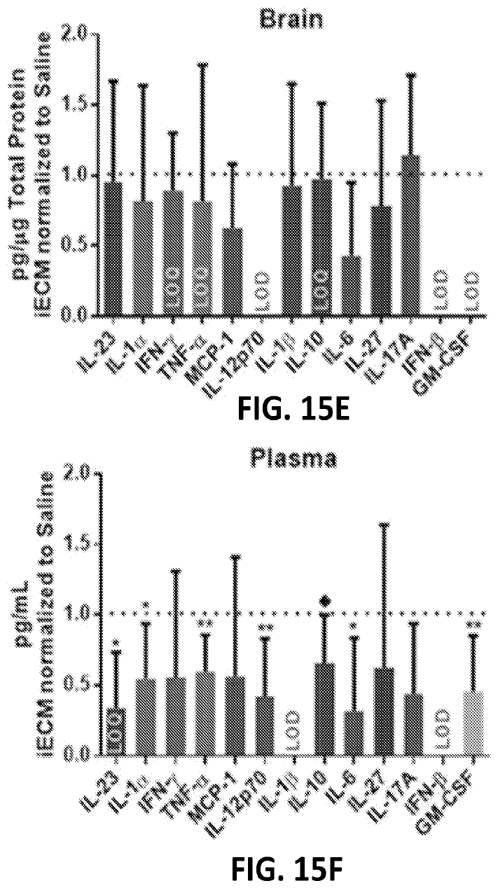

The results are shown in FIGS. 15A to 15F, with *p<0.05, **p<0.01, p <0.1. Spleen homogenate demonstrated significantly decreased expression of IL1b (p=0.049) and trending decreases in TNFa (p=0.052), IL6 (p=0.076), IL27 (p=0.081), and IL17A (p=0.087) (FIG. 15A). Heart homogenate demonstrated significantly decreased expression of IL6 (p=0.040) (FIG. 15B). Kidney homogenate demonstrated significantly decreased expression of IFNg (p=0.007) and IL6 (p=0.014) and trending decrease in IL1b (p=0.077) (FIG. 15C). Lung homogenate demonstrated significantly decreased expression of IL1a (p=0.020) and IL6 (p=0.0064) and trending decreases in IFNy (p=0.077) and IL1b (p=0.097) (FIG. 15D). Brain homogenate did not demonstrate significant or trending results (FIG. 15E). Whole-blood extracted plasma demonstrated the greatest range of significantly decreased cytokine expression, including IL23 (p=0.043), IL1a (p=0.042), TNFa (p=0.0077), IL12p70 (p=0.0083), IL6 (p=0.017), and GM-CSF (p=0.0096) and trending decrease in IL10 (p=0.072) (FIG. 15F).

The decreases of IL1a, IL1b, IL6, IFNy, and TNFa across two or more tissues indicates iECM treatment reduces the expression several pro-inflammatory markers. The lack of significant results in brain tissue may be explained by latent central nervous system immune response and resolution. In mice, serum cytokines may contribute to brain cytokine content over time relative to blood brain barrier permeability and the slower resolution of neural inflammation. This latency could explain the lack of significant differences in cytokine expression results compared to significant gene expression differences identified with Nanostring. In a similar fashion, significant and trending changes in cytokine content in the lung and spleen may not be seen in flow cytometry at the same timepoint because of temporal differences in protein expression and cellular responses like proliferation and migration. Cytokines associated with adaptive immune regulation, particularly IL23, IL27, and IL17A, are more variable among different tissues. IL10, despite being an anti-inflammatory mediator to inhibit secretion of TNFa, IL1b, and IFNy, does not have significant changes in expression other than trending decrease in plasma. However, considering comparisons ratiometrically, IL10 is often elevated in iECM-treated tissues relative to decreased TNFa, IL1b, and/or IFNy.

The plasma cytokine concentration results are of interest to contextualize the translational interest of this therapeutic since numerous studies of human systemic inflammation have assessed plasma or serum cytokine concentrations. In plasma, significantly decreased IL12p70 may be representative of decreased type I helper T cell polarization in the blood. Decreased GM-CSF may be representative of decreased stimulation of granulocytes in the blood. This decrease may be beneficial in certain inflammatory conditions like COVID-19 in which GM-CSF is associated poorer outcomes.

### Example 14: Infusible Extracellular Matrix Generation

An injectable infusible ECM (iECM) solution was generated from porcine left ventricular tissue obtained from adult Yorkshire farm pigs (30-45 kg) based on previously established protocols. In brief, porcine left ventricular tissue was isolated and minced into small pieces. The tissue was decellularized under mechanical agitation in a solution of phosphate buffered saline (PBS) containing 1% (wt/vol) sodium dodecyl sulfate (SDS) (Fischer Scientific, Fair Lawn, NJ) with 0.5% 10,000 U/mL penicillin streptomycin (PS) (Gibco, Life Technologies, Grand Island, NY) for 4-5 days to fully decellularized based on previously established criteria. The decellularized tissue was rinsed for 24 hours with deionized water and received multiple rounds of rinsing in water under high agitation for thoroughly removing residual SDS, lyophilized, and milled into a fine powder. The ECM powder was partially digested with 1 mg/mL pepsin in 0.1M HCl solution for 48 hours before solution was neutralized to pH of 7.4 and reconstituted to physiological salt concentrations.

For infusible ECM generation, the insoluble portion of the digested ECM suspension was pelleted by high-speed centrifugation at 15,000 RCF for 45 minutes at 4° C. The suspended ECM supernatant was transferred into Spectra Por Biotech-Grade CE Dialysis Tubing, 100-500 MWCO (Spectrum Chemical, New Brunswick, NJ), and placed in a series of solutions: 0.5×PBS, 0.25×PBS, and 3 times in sterile deionized water for 12-16 hours. Dialyzed ECM was collected, lyophilized, weighed, and resuspended with 1×PBS for a final concentration of 16 mg/mL based on dry weight. A series of Millipore Stericup vacuum filtrations through 0.45 μm and 0.22 μm PVDF membranes ensured the generated iECM was sterile. iECM was aliquoted, lyophilized, weighed, and stored at −80° C. with desiccate until needed by resuspension in sterile deionized water for a final concentration of 10 mg/mL.

Thus, compositions and methods according to the present disclosure may be used to mitigate or treat a number of conditions including multi-organ damage, vascular injury, leaky vasculature, pulmonary arterial hypertension, lung injury, cardiac injury, or other tissue damage. The present disclosure describes infusible forms of decellularized extracellular matrix hydrogels that can be delivered both intravascularly and via intratracheal instillation to target damaged vascular endothelium and alveolar epithelium of the lungs to promote healing and tissue recovery, validated using a rodent model of lung inflammation. In some embodiments, the present disclosure provides novel therapeutic compositions and methods of use for treatment of COVID-19 patients.

### Aspects

Aspect 1. A method of using infusible extracellular matrix (iECM) as a treatment for vascular injury in a subject, the method including administering to the subject an effective amount of iECM for the treatment, where the iECM contacts the vasculature after the administering.

Aspect 2. The method of Aspect 1, where the vascular injury includes leaky vasculature.

Aspect 3. The method of Aspect 1, where the vascular injury is associated with a tumor, a myocardial infarction, traumatic brain injury, a stroke, or another ischemic condition.

Aspect 4. A method of using an infusible extracellular matrix (iECM) as a treatment for tissue injury associated with damaged vasculature, the method including administering to the subject an effective amount of iECM for the treatment, where the iECM contacts the tissue after the administering.

Aspect 5. The method of Aspect 4, where the tissue injury is associated with one or more of Pulmonary Arterial Hypertension (PAH), use of a ventilator, aspiration of stomach contents, inhalation of environmental toxins, and post-infection complications from a bacterial or viral source.

Aspect 6. The method of Aspect 5, where the aspiration of stomach contents is in the presence or absence of ventilator injury.

Aspect 7. The method of any of Aspects 4 to 6, where the tissue damage includes damage to one or more of lung tissue, heart tissue, kidney tissue, and vascular tissue.

Aspect 8. The method of any of Aspects 1 to 7, where the iECM reduces the infiltration of cells or exudate into the tissue by blocking or reducing vascular permeability.

Aspect 9. The method of any of Aspects 1 to 8, where the iECM reduces the tissue infiltration of reactive oxygen species, inflammatory cytokines, growth factors, exosomes, or any proteins, particles, or molecules by blocking or reducing vascular permeability.

Aspect 10. The method of any of Aspects 1 to 9, where the administering includes delivering via catheter an infusion of a composition including the iECM.

Aspect 11. The method of Aspect 10, wherein the iECM is shear-thinning.

Aspect 12. The method of Aspects 10 or 11, wherein the catheter is a balloon infusion catheter.

Aspect 13. The method of any of Aspects 1 to 12, where a composition including the iECM does not gel in vitro below 38° C.

Aspect 14. The method of any of Aspects 1 to 13, where a composition including the iECM does not gel in the blood after the administering.

Aspect 15. The method of any of Aspects 1 to 14, where a composition including the iECM transitions to a gel form in a tissue after the administering.

Aspect 16. The method of any of Aspects 1 to 15, where a composition including the iECM degrades within one to 14 days after the administering.

Aspect 17. The method of any of Aspects 1 to 16, where a composition including the iECM transitions to form a coating on the endothelium of injured blood vessels after the administering.

Aspect 18. The method of any of Aspects 1 to 17, wherein the iECM is derived from heart, brain, bladder, small intestine, or skeletal muscle tissues, kidney, liver, lung, bronchioles, or blood vessels.

Aspect 19. The method of any of Aspects 1 to 17, wherein the iECM is derived from cardiac tissue.

Aspect 20. The method of any of Aspects 1 to 19, where the iECM includes one of ECM-derived nanofibers, nanorods, and nanoparticles.

Aspect 21. The method of Aspect 20, where the iECM includes nanofibers, nanorods, and nanoparticles less than 0.40 micrometers.

Aspect 22. The method of Aspect 20, where the iECM includes nanofibers, nanorods, and nanoparticles less than 0.22 micrometers.

Aspect 23. The method of any of Aspects 1 to 22, where the iECM is present in a composition having a concentration of 1-20 mg iECM per mL of the composition.

Aspect 24. The method of any of Aspects 1 to 23, where the iECM reduces vascular permeability by binding to exposed ECM in the vasculature through peptides, proteins, or polysaccharides in the iECM.

Aspect 25. The method of Aspect 24, where the iECM binds to the exposed basal lamina.

Aspect 26. The method of Aspect 24, where the iECM binds to exposed receptors on endothelial cells including integrins, selectins, and/or other transmembrane receptors.

Aspect 27. The method of any of Aspects 1 to 26, where the iECM reduces vascular permeability by binding to leaky or inflamed vasculature in an area of shear stress <1000 s⁻¹.

Aspect 28. The method of Aspect 27, wherein platelets bind to the iECM.

Aspect 29. The method of Aspect 27, wherein the iECM attracts endothelial or vascular progenitor cells.

Aspect 30. A method of preparing infusible extracellular matrix (ECM), including fractionating ECM for infusion.

### REFERENCES

1. van de Veerdonk M C, Kind T, Marcus J T, Mauritz G J, Heymans M W, Bogaard H J, Boonstra A, Marques K M, Westerhof N, Vonk-Noordegraaf A. Progressive right ventricular dysfunction in patients with pulmonary arterial hypertension responding to therapy. J Am Coll Cardiol. 2011; 58:2511-9.
2. Rabinovitch M. Molecular pathogenesis of pulmonary arterial hypertension. J Clin Invest. 2012; 122:4306-13.
3. Suen C M, Mei S H, Kugathasan L, Stewart D J. Targeted delivery of genes to endothelial cells and cell- and gene-based therapy in pulmonary vascular diseases. Compr Physiol. 2013; 3:1749-79.
4. Matthay M A, Zemans R L, Zimmerman G A, Arabi Y M, Beitler J R, Mercat A, Herridge M, Randolph A G, Calfee C S. Acute respiratory distress syndrome. Nat Rev Dis Primers. 2019; 5:18.
5. Modrykamien A M, Gupta P. The acute respiratory distress syndrome. Proc (Bayl Univ Med Cent). 2015; 28:163-71.
6. Hepokoski M, Englert J A, Baron R M, Crotty-Alexander L E, Fuster M M, Beitler J R, Malhotra A, Singh P. Ventilator-induced lung injury increases expression of endothelial inflammatory mediators in the kidney. Am J Physiol Renal Physiol. 2017; 312:F654-F660.
7. Acute Respiratory Distress Syndrome N, Brower R G, Matthay M A, Morris A, Schoenfeld D, Thompson B T, Wheeler A. Ventilation with lower tidal volumes as compared with traditional tidal volumes for acute lung injury and the acute respiratory distress syndrome. N Engl J Med. 2000; 342:1301-8.
8. Choi W I, Quinn D A, Park K M, Moufarrej R K, Jafari B, Syrkina O, Bonventre J V, Hales C A. Systemic microvascular leak in an in vivo rat model of ventilator-induced lung injury. Am J Respir Crit Care Med. 2003; 167:1627-32.
9. Inciardi R M, Lupi L, Zaccone G, et al. Cardiac Involvement in a Patient With Coronavirus Disease 2019 (COVID-19). JAMA Cardiology. 2020.
10. Mao L, Wang M, Chen S, He Q, Chang J, Hong C, Zhou Y, Wang D, Li Y, Jin H, Hu B. Neurological Manifestations of Hospitalized Patients with COVID-19 in Wuhan, China: a retrospective case series study. medRxiv. 2020:2020.02.22.20026500.
11. Wu Y, Xu X, Chen Z, Duan J, Hashimoto K, Yang L, Liu C, Yang C. Nervous system involvement after infection with COVID-19 and other coronaviruses. Brain, Behavior, and Immunity. 2020.
12. Wassenaar J W, Gaetani R, Garcia J J, Braden R L, Luo C G, Huang D, DeMaria A N, Omens R I, Christman K L. Evidence for Mechanisms Underlying the Functional Benefits of a Myocardial Matrix Hydrogel for Post-M I Treatment. Journal of the American College of Cardiology. 2016; 67:1074-1086.
13. Wang R M, Johnson T D, He J, Rong Z, Wong M, Nigam V, Behfar A, Xu Y, Christman K L. Humanized mouse model for assessing the human immune response to xenogeneic and allogeneic decellularized biomaterials. Biomaterials. 2017; 129:98-110.
14. Meng F W, Slivka P F, Dearth C L, Badylak S F. Solubilized extracellular matrix from brain and urinary bladder elicits distinct functional and phenotypic responses in macrophages. Biomaterials. 2015; 46:131-140.
15. Spang M T, Lazerson T S, Bhatia S, Corbitt J, Sandoval G, Luo C, Osborn K G, Cabrales P, Kwon E, Contijoch F, Reeves R R, DeMaria A N, Christman K L. A New Form of Decellularized Extracellular Matrix Hydrogel for Treating Ischemic Tissue via Intravascular Infusion. bioRxiv. 2020:2020.04.10.028076.
16. Slivka P F, Dearth C L, Keane T J, Meng F W, Medberry C J, Riggio R T, Reing J E, Badylak S F. Fractionation of an ECM hydrogel into structural and soluble components reveals distinctive roles in regulating macrophage behavior. Biomaterials Science. 2014; 2:1521-1534.
17. Seif-Naraghi S B, Singelyn J M, Salvatore M A, et al. Safety and efficacy of an injectable extracellular matrix hydrogel for treating myocardial infarction. Sci Transl Med. 2013; 5:173ra25.

1. A method of using infusible extracellular matrix (iECM) as a treatment for vascular injury in a subject, the method comprising administering to the subject an effective amount of iECM for the treatment, wherein the iECM contacts the vasculature after the administering.

2. The method of claim 1, wherein the vascular injury comprises leaky vasculature.

3. The method of claim 1, wherein the vascular injury is associated with a tumor, a myocardial infarction, traumatic brain injury, a stroke, or another ischemic condition.

4. A method of using an infusible extracellular matrix (iECM) as a treatment for tissue injury associated with damaged vasculature, the method comprising administering to the subject an effective amount of iECM for the treatment, wherein the iECM contacts the tissue after the administering.

5. The method of claim 4, wherein the tissue injury is associated with one or more of Pulmonary Arterial Hypertension (PAH), use of a ventilator, aspiration of stomach contents, inhalation of environmental toxins, and post-infection complications from a bacterial or viral source.

6. The method of claim 4, wherein the tissue damage comprises damage to one or more of lung tissue, heart tissue, kidney tissue, and vascular tissue.

7. The method of claim 1, wherein the iECM reduces the infiltration of cells or exudate into a tissue, or tissue infiltration of reactive oxygen species, inflammatory cytokines, growth factors, exosomes, or any proteins, particles, or molecules, by blocking or reducing vascular permeability.

8. The method of claim 1, wherein the administering comprises delivering via catheter an infusion of a composition comprising the iECM.

9. The method of claim 1, wherein a composition comprising the iECM one or more of (1) does not gel in vitro below 38° C., (2) does not gel in the blood after the administering, (3) transitions to a gel form in a tissue after the administering, (4) degrades within one to 14 days after the administering, or (5) transitions to form a coating on the endothelium of injured blood vessels after the administering.

10. The method of claim 1, wherein the iECM is derived from heart, brain, bladder, small intestine, or skeletal muscle tissues, kidney, liver, lung, bronchioles, or blood vessels.

11. The method of claim 1, wherein the iECM is derived from cardiac tissue.

12. The method of claim 1, wherein the iECM comprises one of ECM-derived nanofibers, nanorods, and nanoparticles.

13. The method of claim 1, wherein the iECM is present in a composition having a concentration of 1-20 mg iECM per mL of the composition.

14. The method of claim 1, where the iECM reduces vascular permeability by binding to exposed ECM in the vasculature through peptides, proteins, or polysaccharides in the iECM.

15. A method of preparing infusible extracellular matrix, the method comprising fractionating extracellular matrix for infusion.
